(19) United States
(12) Patent Application Publication
(10) Pub. No.: US 2005/0020536 A1
(43) Pub. Date: Jan. 27, 2005

(54) Highly pure fondaparinux sodium composition, process for preparing said composition and pharmaceutical compositions containing it as active principle

(76) Inventors: Jean-Francois Branellec, Mont Saint Aignan (FR); Christian Morello, Clapiers (FR); Pierre Potier, Noyers sur Jabron (FR); Patrick Trouilleux, Sisteron (FR); Petrus Marcus Franciscus Maria Bastiaansen, Diosynth (NL); Henricus Cornelis Jozephus Claassen, Rijkevoort (NL)

Correspondence Address: SANOFI-SYNTHELABO INC., 9 GREAT VALLEY PARKWAY, P.O. BOX 3026, MALVERN, PA, 19355, US

(21) Appl. No.: 10/881,250

(22) Filed: Jun. 30, 2004

Related U.S. Application Data
(63) Continuation-in-part of application No. 10/375,268, filed on Feb. 27, 2003.

(30) Foreign Application Priority Data
Feb. 12, 2004 (WO) PCT/FR04/00314

(51) Int. Cl. A61K 31/737

(52) U.S. Cl. 514/54; 536/54

(57) ABSTRACT

The present invention relates to a highly pure fondaparinux sodium composition, to a process for preparing said composition for its use in pharmaceutical compositions, and also to the pharmaceutical compositions containing it as active principle.

This application is a continuation-in-part of prior copending application Ser. No. 10/375,268 filed Feb. 27, 2003.

The present invention relates to a highly pure fondaparinux sodium composition, to a process for preparing said composition for its use in pharmaceutical compositions, and also to the pharmaceutical compositions containing it as active principle.

Fondaparinux sodium, or methyl O-2-deoxy-6-O-sulfo-2-(sulfoamino)-α-D-glucopyranosyl-(1→4)-O-β-D-glucopyranuronosyl-(1→4)-O-2-deoxy-3,6-di-O-sulfo-2-(sulfoamino)-α-D-glucopyranosyl-(1→4)-2-O-sulfo-α-D-glucopyranosyl-(1→4)-O-2-O-sulfo-L-idopyranurosyl-(1→4)-2-deoxy-6-O-sulfo-2-(sulfoamino)-α-D-glucopyranoside, decakissodium salt, compound of formula I 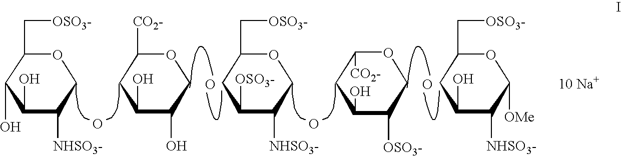
is a compound with very powerful anti-factor Xa (anti Xa) activity and very advantageous antithrombotic properties. This compound, which has a molecular mass of 1728, is of use in the treatment and prevention of thromboembolic diseases and it is the active principle in the specialty product Arixtra®, which is administered subcutaneously.

This compound is obtained according to the process described in patents EP 084 999 and U.S. Pat. No. 4,818,816. Fondaparinux sodium is derived from a chemical synthesis comprising more than 50 steps. This process makes it possible to obtain crude fondaparinux sodium, which is a mixture consisting of fondaparinux sodium and other related oligosaccharides. The fondaparinux sodium content of this mixture, evaluated by anion exchange high performance liquid chromatography (HPLC), is approximately 70%.

Several steps of purification by column chromatography and by precipitation are necessary in order to obtain fondaparinux sodium having a purity which does not exceed 96.0%.

Furthermore, the large number of steps for synthesis makes it very difficult to standardize industrial batches.

Given the complexity of the structure of fondaparinux sodium and of its synthesis intermediates, many impurities can form in the course of the synthesis. In addition, the slightest variation in the operating conditions results in batches of crude fondaparinux sodium being obtained which contain related products in considerable amounts. These related products, which do not have anti-Xa activity or which have very slight activity, have a chemical structure and physicochemical characteristics which are very similar to fondaparinux sodium, and cannot be eliminated satisfactorily by purification methods indicated above. Moreover, it has been observed that some of these products are readily degradable, when they are subjected to sterilization by methods such as autoclaving, and thus produce additional impurities.

Fondaparinux sodium, the active principle in a pharmaceutical specialty product, must satisfy certain quality criteria and standards and must in particular be as highly pure as possible. As a result, industrial batches which contain related products in considerable amounts cannot be used for preparing pharmaceutical specialty products. Thus, it is important to have highly pure fondaparinux sodium compositions, and in particular industrial amounts of such compositions, and also a process for obtaining them.

Surprisingly, it has now been found that, by subjecting crude fondaparinux sodium to at least one step of purification on activated charcoal and then to one or more conventional steps for purifying sugars, such as column chromatography or precipitation/crystallization, it is possible to obtain highly pure fondaparinux sodium. These are compositions consisting mainly of fondaparinux sodium and other oligosaccharides such as penta- or octasaccharides, in particular related pentasaccharides, and which contain at least 97% of fondaparinux sodium.

According to the invention, these compositions are of great value from an industrial point of view, in particular in the pharmaceutical domain. They satisfy a pharmaceutical quality and can be used as active principle in medicinal products.

Surprisingly, it has also been found that simply the act of being passed over activated charcoal makes it possible to obtain compositions of well-determined constitution, from batches of crude fondaparinux sodium having very different contents of fondaparinux sodium and related impurities.

Activated charcoal has for a long time been used to remove impurities from gases and liquids. For example, activated charcoal is used to purify waste water and drinking water. Activated charcoal is also used to remove traces of impurities with a very different structure compared to the product to be purified.

In the field of sugar derivatives, activated charcoal has been used to purify glucosides. For example, patent application FR 2 557 139 describes a process for purifying crude glucosides in solution, using a yeast, which makes it possible to decrease mono- or oligosaccharide reducing sugars. According to this process, the activated charcoal is used in the final stages to discolour and deodorize the aqueous solution of glucoside free of reducing oligosaccharides.

Moreover, patent application FR 2 732 024 describes a process for purifying chemically modified cyclodextrins using activated charcoal in a liquid phase. According to this process, only activated charcoal of specific origin, in particular that obtained from coal and from coconut husks, makes it possible to remove the organic impurities produced during the chemical modification of cyclodextrins, and also the residual reaction solvents.

Known processes using activated charcoal which make it possible to purify substances by eliminating large amounts of impurities having a structure similar to the substance to be purified are rare. Patent application WO 01/16079, which describes a process for purifying 2,6-naphthalenecarboxylic acid which uses activated charcoal and a specific material, is for example known. This process makes it possible to remove the impurities from synthesis, in particular 1-naphthanoic acid and 2-naphthanoic acid. According to this process, a solution of the product to be purified is filtered over microbeads of absorbent which contain activated charcoal.

The present invention relates to highly pure fondaparinux sodium compositions, including those which contain a small percentage of impurities which are related oligosaccharides.

Analysis by anion exchange high performance liquid chromatography (HPLC) and detection by UV at λ=210 nm of the purified fondaparinux sodium obtained according to the process described in patents EP 084 999 and U.S. Pat. No. 4,818,816 has made it possible to identify a certain number of impurities which are related oligosaccharides. The main impurities identified are as follows:

Impurity A (Relative Retention Time Relative to the Retention time of Fondaparinux Sodium (Rrt)=0.8)

Methyl (2-amino-2-deoxy-6-O-sodium sulfonato-α-D-glucopyranosyl)-(1→4)-(sodium β-D-glucopyranosyluronate)-(1→4)-(2-deoxy-2-sodium sulfoamino-3,6-di-O-sodium sulfonato-α-D-glucopyranosyl)-(1→4)-(sodium 2-O-sodium sulfonato-α-L-idopyranosylonate)-2-deoxy-2-sodium sulfoamino-6-O-sodium sulfonato-α-D-glucopyranoside, compound of formula II: 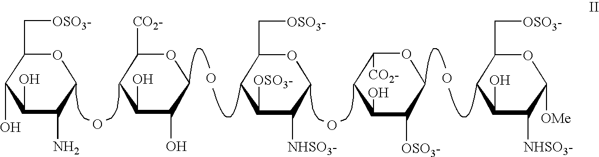
Impurity B (Rrt=0.93)

It is a mixture consisting mainly of the impurity methyl (2-deoxy-2-sodium sulfoamino-6-O-sodium sulfonato-α-D-glucopyranosyl)-(1→4)-(sodium β-D-glucopyranosyluronate)-(1→4)-(2-deoxy-2-sodium sulfoamino-3,6-di-O-sodium sulfonato-α-D-gluocopyranosyl)-(1→4)-(sodium 2,3-di-O-sodium sulfonato-α-L-idopyranosyluronate)-2-deoxy-2-sodium sulfoamino-6-O-sodium sulfonato-α-D-glucopyranoside, compound of formula III: 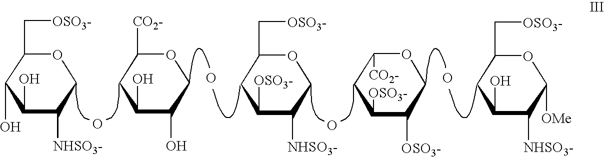
and of the impurity methyl (2-deoxy-2-formylamino-6-O-sodium sulfonato-α-D-glucopyranosyl)-(1→4)-(sodium β-D-glucopyranosyluronate)-(1→4)-(2-deoxy-2-sodium sulfoamino-3,6-di-O-sodium sulfonato-α-D-gluocopyranosyl)-(1→4)-(sodium 2-O-sodium sulfonato-α-L-idopyranosyluronate)-2-deoxy-2-sodium sulfoamino-6-O-sodium sulfonato-α-D-glucopyranoside, compound of formula IV: 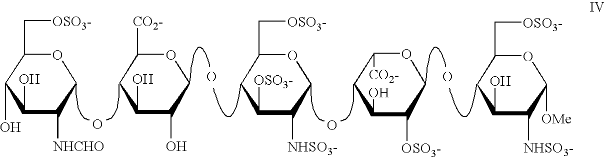
the latter occurring in the mixture in lesser amounts, or often being absent.
Impurity C (Rrt=1.2)

It is a mixture consisting of methyl (2-deoxy-2-sodium sulfoamino-6-O-sodium sulfonato-α-D-glucopyranosyl)-(1→4)-(sodium 2-O-cyclohexylmethyl-β-D-glucopyranosyluronate)-(1→4)-(2-deoxy-2-sodium sulfoamino-3,6-di-O-sodium sulfonato-α-D-gluocopyranosyl)-(1→4)-(sodium 2-O-sodium sulfonato-α-L-idopyranosyluronate)-2-deoxy-2-sodium sulfoamino-6-O-sodium sulfonato-α-D-glucopyranoside, compound of formula V: 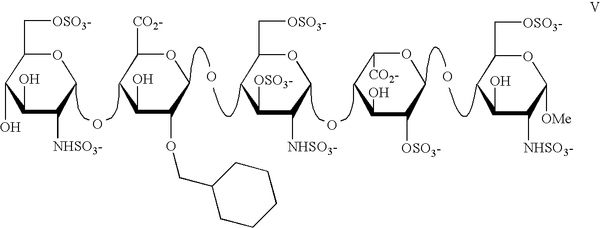
Impurity D (Rrt=1.3)

It is methyl (sodium 4-O-(sodium 7-hydroxy-2-oxo-6-[(sulfonatooxy)methyl]hexahydro-4-H-pyrano[3,4-d][1,3]oxazol-4-yl}-β-D-glucopyranosyluronate)-(1→4)-(2-deoxy-2-sodium sulfoamino-3,6-di-O-sodium sulfonato-β-D-gluocopyranosyl)-(1→4)-(sodium 2, O-sodium sulfonato-α-L-idopyranosyluronate)-2-deoxy-2-sodium sulfoamino-6-O-sodium sulfonato-α-D-glucopyranoside, compound of the formula VI: 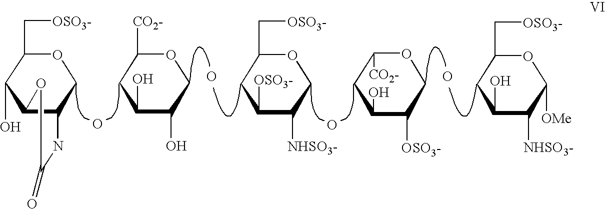
Impurity E (Rrt=1.4)

It is methyl (3,4-di-O-[(2-deoxy-2-sodium sulfoamino-6-O-sodium sulfonato-α-D-glucopyranosyl)-(1→4)-(sodium β-D-glucopyranosyluronate)-(1→4)-2-deoxy-2-sodium sulfoamino-3,6-di-O-sodium sulfonato-α-D-gluocopyranosyl]-2-O-sodium sulfonato-α-L-idopyranosyluronate)-2-deoxy-2-sodium sulfoamino-6-O-sodium sulfonato-α-D-glucopyranoside, compound of formula VII: 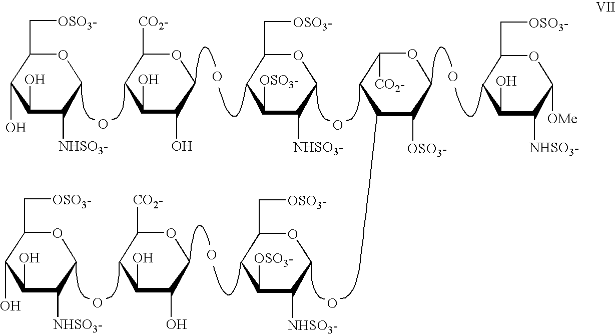
Impurity F (Rrt=1.5)

It is methyl (2-deoxy-2-sodium sulfoamino-6-O-sodium sulfonato-α-D-glucopyranosyl-(1→4)-(sodium β-D-glucopyranosyluronate)-(1→4)-(2-deoxy-2-sodium sulfoamino-3,6-di-O-sodium sulfonato-α-D-glucopyranosyl)-(1→4)-(sodium 2-O-sodium sulfonato-α-L-idopyranosyluronate)-2-benzamido-2-deoxy-6-O-sodium sulfonato-α-D-glucopyranoside, compound of formula VIII: 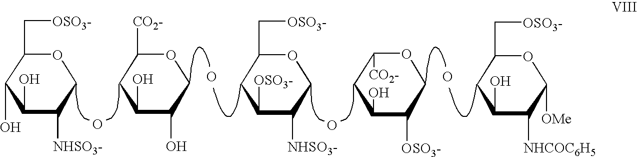
Impurity G (Rrt=1.58)

It is methyl (2-deoxy-2-sodium sulfoamino-6-O-sodium sulfonato-α-D-glucopyranosyl)-(1→4)-(sodium 3-O-cyclohexylmethyl-β-D-glucopyranosyluronate)-(1→4)-(2-deoxy-2-sodium sulfoamino-3,6-di-O-sodium sulfonato-β-D-glucopyranosyl)-(1→4)-(sodium 2-O-sodium sulfonato-α-L-idopyranosyluronate)-2-deoxy-2-sodium sulfoamino-6-O-sulfonato-α-D-glucopyranoside, compound of formula IX: 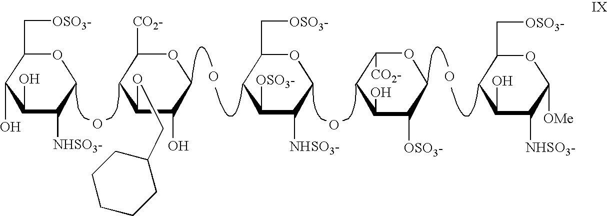
Impurity H (Rrt=1.60)

It is methyl (2-deoxy-4-O-{disodium 3,4,8-trihydroxy-11-oxo-9-(sulfonatooxy)-7-[(sulfonatooxy)methyl]decahydro-2H,5aH-dipyrano[2,3-b:2,3-f][1,4]oxazepin-2-yl}-2-sodium sulfoamino-3,6-di-O-sodium sulfonato-α-D-glucopyranosyl)-(1→4)-(sodium 2-O-sodium sulfonato-α-L-idopyranosyluronate)-2-deoxy-2-sodium sulfoamino-6-O-sulfonato-α-D-glucopyranoside, compound of formula X:

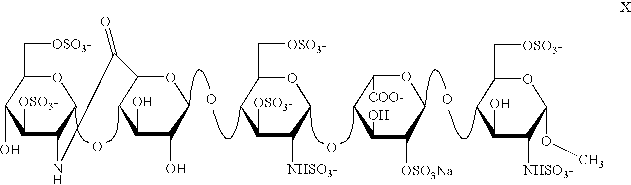

The impurities indicated above were identified by mass spectrometry and/or NMR spectrometry.

Since the extinction coefficient for these compounds is not evaluated with certainty, the amount of these compounds in the crude fondaparinux sodium or in the fondaparinux sodium compositions according to the invention is expressed as percentage (%) according to the following formula:
[Aᵢ/Aₜₒₜₐₗ]×100
where
Aᵢ: area of the peak corresponding to the related substance “i” observed on the chromatogram obtained with the solution to be analyzed.
Aₜₒₜₐₗ: sum of the areas of the peaks observed in the test solution.

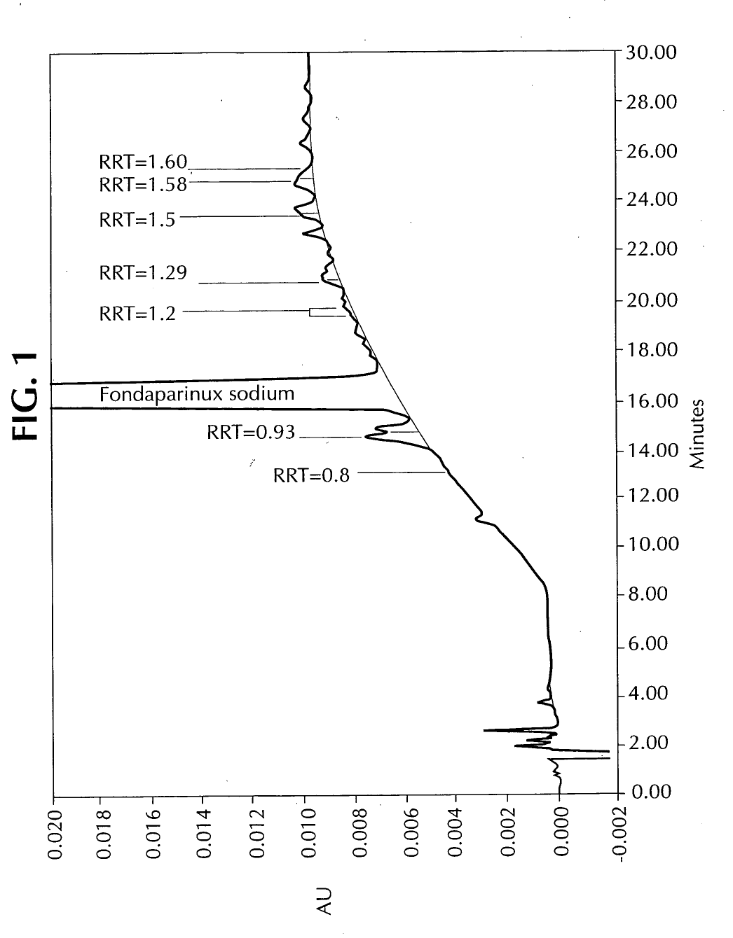

FIG. 1 is an anion exchange HPLC chromatogram showing the retention times of the oligosaccharide impurities relative to that of fondaparinux sodium.

The present invention relates to a highly pure fondaparinux sodium composition which contains at least 97%, preferably at least 98%, of fondaparinux sodium, the remainder of the components being related oligosaccharides, it being understood that the amount of fondaparinux sodium in these compositions never corresponds to 100%. Among these compositions, preference is given to the compositions for which the amount of impurity B is at most 0.8% and, in particular among the latter compositions, preference is given to those for which the amount of impurity D is at most 1.0%, preferably 0.5%.

The present invention relates more particularly to a highly pure fondaparinux sodium composition which contains at least 98% of fondaparinux sodium, the remainder of the components being related oligosaccharides, it being understood that the amount of fondaparinux sodium in these compositions never corresponds to 100%, the amount of impurity B being at most 0.8%, the amount of impurity C being at most 1.0% and the amount of impurity D being at most 0.5%. Among these compositions, preference is given to the compositions for which the amount of impurity B is at most 0.8%, the amount of impurity C is at most 0.6% and the amount of each of the related oligosaccharide impurities, other than impurities B and C, is at most 0.3%.

The present invention also relates more particularly to a highly pure fondaparinux sodium composition which contains at least 98% of fondaparinux sodium, the remainder of the components being related oligosaccharides, it being understood that the amount of fondaparinux sodium in these compositions never corresponds to 100%, the amount of impurity B being at most 0.8%, the amount of impurity D being at most 0.5%, and the amount of each of the related oligosaccharide impurities other than impurities B and D being at most 0.3%. Among these compositions, preference is given to the compositions for which the amount of impurity D is at most 0.3%, and in particular preference is given to the compositions for which the amount of impurity B is at most 0.5% and the amount of impurity D is at most 0.3%.

All these compositions are preferably in industrial amounts, more precisely in amounts of 5 g to 8000 g, for example of 10 g, of 100 g, 500 g, 800 g, 1000 g, 2000 g or 5000 g, and can be obtained from crude fondaparinux sodium.

As indicated above, the fondaparinux sodium is derived from a chemical synthesis of oligosaccharides, comprising more than 50 steps. This process makes it possible to obtain crude fondaparinux sodium, which is approximately 70% pure by ionic HPLC.

To obtain the compositions of the present invention, the crude fondaparinux is subjected to the action of activated charcoal. This step, surprisingly, makes it possible to substantially decrease the amount of impurities present, and in particular impurity D, a compound with a structure similar to fondaparinux sodium, but which degrades very readily during the various steps for sterilizing the pharmaceutical specialty products having fondaparinux sodium as active principle.

The compositions thus obtained are then purified with conventional methods, used until now for oligosaccharides, namely purification on a chromatography column and precipitation with ethanol. Highly pure fondaparinux sodium compositions in which the content of this active principle is at least 97% are thus obtained.

Compositions of fondaparinux sodium having undergone at least one step of purification on activated charcoal are also part of the invention.

The present invention also relates to a process for purifying fondaparinux sodium, which comprises at least one step of purification on activated charcoal.

The activated charcoal used to obtain the compositions according to the invention can be produced from various compounds, such as plants (wood, peat, coconut shell or coffee bean), minerals (charcoal, coal or hydrocarbons) or animals (blood, bone, etc.).

These charcoals referred to as activated charcoals are activated either by the action of steam, or by chemical action. The porosity which results therefrom then determines the absorption properties of the charcoal and confers on it its effectiveness. According to the present invention, steam-activated charcoal of plant origin is preferably used. Plant charcoal of the type Norit A Supra Euro® produced by Norit is preferred.

Activated charcoal of type 3S® provided by CECA, of type CPL® provided by CECA, of type GAC 1240® provided by Norit or of type 4SC provided by CECA is also preferred.

The purification on activated charcoal can be carried out on the crude fondaparinux sodium in aqueous solution, before precipitation with ethanol or after precipitation with ethanol. The purification on activated charcoal is preferably carried out after having performed a first purification by precipitation with ethanol.

The treatment with activated charcoal can be carried out either in a batch system stirred with pulverulent charcoal, or in a dynamic system with charcoal attached to filtering plates.

Treatment in a dynamic system is preferred.

The ratio of activated charcoal to fondaparinux sodium is between 50% and 150%, preferably between 80% and 125%, or more particularly 100%.

In the two situations, i.e. treatment with activated charcoal or in a dynamic system on a filter, the purification is carried out at a temperature of between 5° C. and 50° C., advantageously between 15° C. and 35° C., and preferably at ambient temperature.

The pH of the solution of crude fondaparinux to be purified is adjusted, if necessary, to between 5 and 10, preferably to between 7 and 9.

The primary filtrate and also the washing solutions are pooled and filtered over a membrane with a porosity of less than 1 μm, advantageously less than 0.22 μm.

The present invention also relates to pharmaceutical compositions containing, as active principle, a composition of sodium fondaparinux purified according to the invention. These compositions are preferably in the form of injectable solutions for subcutaneous or parenteral administration.

The following non-limiting examples illustrate the invention:

Determination of the Fondaparinux Sodium Titre in the Compositions

The fondaparinux sodium titre is determined by ionic HPLC and detection by UV at λ=210 nm.

For the high performance liquid chromatography, the following are used: a) an anion exchange column, 250 mm in length and 4 mm in internal diameter, filled with a polymeric matrix onto which are grafted latex microbeads bearing quaternary ammonium functional groups (Dionex Carbopac® ref 035391 or equivalent) and b) an anion exchange precolumn, 50 mm in length and 4 mm in internal diameter, filled with the same stationary phase as the analytical column (Dionex Carbopac® ref 043096 or equivalent). The column temperature is kept at 30° C. during the analysis.

As mobile phase, a mixture of mobile phases A (150 μl of a solution of dimethyl sulfoxide diluted to 1/10 in 1000 ml of H₂O) and B (117 g of NaCl dissolved in 1000 ml of H₂O) is used.

The flow rate of the mobile phase is adjusted to 1 ml/min and the following linear gradient is used:

| Time (min) | % mobile phase A | % mobile phase B |
|---|---|---|
| 0 | 50 | 50 |
| 5 | 50 | 50 |
| 25 | 10 | 90 |
| 30 | 10 | 90 |
| 35 | 50 | 50 |
| 50 | 50 | 50 |

The conformity of the system is evaluated with a reference solution containing the major impurities. A standard chromatogram is given in FIG. 1.

The peak for impurity B (Rrt=0.93) and the peak eluting just after this peak should appear as two distinct peaks.

The peak for impurity D and the peak for Rrt=1.29 should appear as two distinct peaks.

The peak for impurity G (Rrt=1.58) and the peak for impurity H (Rrt=1.60) should appear as two distinct peaks.

A solution of a composition of fondaparinux sodium, with a known fondaparinux sodium content which is approximately 10 mg/ml, is used as reference solution (RS). A solution containing 100 mg of the composition to be analyzed, in 10 ml of H₂O, is used as test solution (S2).

The concentration of fondaparinux sodium in the composition to be analyzed, free of water and of solvents, is calculated according to the following formula:
Cref(a)×[S(a)/Sref(a)]×V/m×100×[100/100−(W+S)]
where
Cref(a): concentration of fondaparinux sodium (mg/ml) in the reference solution RS
S(a) and Sref(a): areas of the peaks of fondaparinux sodium obtained respectively with the test solution S2 and the reference solution RS
V: volume for dissolving the test sample for preparing the test solution S2 (ml)
m: test sample for the substance to be analyzed, for preparing the test solution S2
W: water content of the substance to be analyzed (%)
S: solvent content of the substance to be analyzed (%).

The content of the related substances in the solution to be analyzed of the composition to be analyzed, free of water and of solvents, is calculated according to the following formula:
[Aᵢ/Aₜₒₜₐₗ]×100
where
Aᵢ: area of the peak corresponding to the related substance “i” observed on the chromatogram obtained with the test solution S2,
Aₜₒₜₐₗ: sum of the areas of the peaks observed in the test solution S2.

### EXAMPLE 1

Obtaining a Composition According to the Invention

Step A: Purification of the Crude Fondaparinux Sodium—Stirred Batchwise Charcoal Treatment

For the treatment in a stirred batchwise system, the crude fondaparinux sodium is dissolved in demineralized water in a proportion of 40 to 60 g of pure fondaparinux sodium per litre.

The batch of crude fondaparinux sodium used for this test contains 71.4% of fondaparinux sodium.

The amount of impurity A is equal to 1.5%,

the amount of impurity B is equal to 1.7%,
the amount of impurity C is equal to 0.7%,
the amount of impurity D is equal to 1.6%,
the amount of impurity F is equal to 4.5%,
the amount of impurity G is equal to 0.3%,
and the amount of impurity H is 0.5%.

The pH of the solution is adjusted to 7.6.

The content of crude fondaparinux sodium salts is sufficient to guarantee a conductivity in solution enabling the action of the charcoal treatment. However, if necessary, the conductivity may be adjusted, by introducing powdered sodium chloride, to between 30 and 100 mS/cm.

Vegetable black (powdered Norit A Supra Euro®) is added at 100%.

The medium is stirred for between 2 and 4 hours, preferably 3 hours, and then filtered.

The charcoal is then washed with a solution of water/sodium chloride with a conductivity of 50 mS/cm.

This washing is carried out in two steps, with volumes equal to half of the volume of the solution initially used, with re-impasting of the charcoal and filtration.

The primary filtrate and also the washing solution are then pooled and filtered through a membrane with a porosity of 0.22 mm.

The composition thus obtained contains 94% of fondaparinux sodium.

The amount of impurity A is less than 0.1%,

the amount of impurity B is equal to 0.6%,
the amount of impurity C is less than 0.1%,
the amount of impurity D is equal to 0.2%,
the amount of impurity F is less than 0.1%,
the amount of impurity G is less than 0.1%,
and the amount of impurity H is less than 0.1%.
Yield: 90%.
   Step B: Purification by Anion Exchange Chromatography

The fondaparinux sodium composition obtained in the preceding step is then purified by anion exchange chromatography on a Sepharose Q Fast Flow column. The column is equilibrated with 0.2 M sodium chloride (NaCl). The fondaparinux sodium composition is dissolved in water and the conductivity is adjusted with water or NaCl so as to be less than 20 mS/cm.

The product to be purified is loaded at a rate of 15 g of fondaparinux sodium per litre of gel. The column feed is rinsed with 0.2 M NaCl, and the column is then washed with a 0.46 M NaCl solution, which makes it possible to remove the weakly charged impurities.

The fondaparinux sodium is then eluted with a 0.8 M NaCl solution, the column is then regenerated, and the highly charged impurities are desorbed with a 2.00 M NaCl solution.

Fractions are analyzed and the fractions having a purity of greater than or equal to 95% are pooled.

The purified solutions containing fondaparinux sodium are concentrated so as to obtain solutions containing 20 to 70 g/l of fondaparinux sodium. The conductivity is adjusted with water or NaCl so as to be from 45 to 90 mS/cm. The solution thus obtained is filtered through a microfiltration membrane, and then mixed with ethanol, at a ratio of 1:5 V/V.

A highly pure fondaparinux sodium composition having the following characteristics is thus obtained by precipitation:
fondaparinux sodium content greater than 99.8%,
impurity A content less than 0.1%,
impurity B content less than 0.2%,
impurity C content less than 0.1%,
impurity D content less than 0.1%,
content of impurities F, G and H less than 0.1%,
cumulative yield: 90.4%.

### EXAMPLE 2

Purification of the Crude Fondaparinux Sodium by Column Chromatography and Precipitation (without Purification on Charcoal)

Step A

The batch of crude fondaparinux used for testing is that described in example 1.

This batch was subjected to purification by column chromatography according to the conditions described in example 1, step B.

Step B

This purification was followed by second purification on a Sepharose Q Fast Flow column according to the following conditions:

The column was equilibrated with a 0.4 M NaCl solution. The fondaparinux sodium composition to be purified was diluted in water and the conductivity was adjusted with water or NaCl so as to be less than 35 mS/cm. The fondaparinux sodium solution was loaded at a rate of 12 to 15 g of fondaparinux sodium per litre of gel.

The column feed line was rinsed with 0.4 M NaCl and the column was then rinsed with a 0.48 M NaCl solution (desorption of the weakly charged impurities). Next, the fondaparinux was eluted with a 0.75 M NaCl solution, the column was then regenerated, and the highly charged impurities were desorbed with a 2.00 M NaCl solution.

The various fractions were analyzed, and those having a purity greater than or equal to 95% were then pooled.

Next, a nanofiltration was carried out, followed by microfiltration and then a precipitation as described in example 1, step B.

The fondaparinux sodium composition thus obtained has the following characteristics:

Fondaparinux sodium content 96.0%,

impurity A content less than 0.1%,
impurity B content less than 0.2%,
impurity C content 0.7%,
impurity D content 1.4%,
impurity F content 1.2%,
impurity G content less than 0.2%,
impurity H content 0.6%,
cumulative yield 88.5%.

### EXAMPLE 3

Obtaining a Composition According to the Invention

Step A: Purification of the Crude Fondaparinux Sodium—Dynamic Treatment on Activated Charcoal

For the dynamic treatment, the preparation of the solution containing crude fondaparinux sodium is identical to that described in example 1, step A. The solution of fondaparinux sodium used has a fondaparinux sodium concentration of 50 g per litre of demineralized water. The pH is adjusted to between 5 and 10 and the conductivity of the solution is adjusted to between 30 and 100 mS/cm.

The activated charcoal is, in this case, immobilized in a filter, using a resin-type adjuvant, between two cellulose plates (CUNO filter). The flow rate for percolation through the filter is 1000 l/h/m². The solution is recycled for two hours.

The washing is carried out with an NaCl solution having a conductivity of 50 mS/cm and a volume corresponding to 80% of the initial volume used. Three successive washes are carried out.

Next, the primary filtrate and also the washing solutions are pooled and filtered through a membrane with a porosity of less than 1 μm.

The batch of crude fondaparinux sodium used for this test contains 73% of fondaparinux sodium.

The amount of impurity A is equal to 3.9%,

the amount of impurity B is equal to 3.0%,
the amount of impurity C is equal to 0.7%,
the amount of impurity D is equal to 0.9%,
the amount of impurity F is equal to 0.3%,
the amount of impurity G is equal to 0.4%,
and the amount of impurity H is 0.3%.
   Amount of other unidentified related oligosaccharides: approximately 1%.

After the treatment on activated charcoal, the fondaparinux sodium composition is as follows:

Fondaparinux sodium content 90.7%,

impurity A content less than 0.2%,
impurity B content 2.1%,
impurity C content less than 0.2%,
impurity D content 0.3%,
impurity F content less than 0.1%,
impurity G content less than 0.1%,
impurity H content less than 0.1%,
yield: 93.1%.
   Step B: Purification by Anion Exchange Chromatography

The procedure as described in example 1, step B is then carried out. After analysis of the fractions, the fractions having a purity greater than 70% are pooled.

At the end of this step, a highly pure fondaparinux sodium composition which has the following characteristics is obtained:

Fondaparinux sodium content 99.1%,

impurity A content less than 0.1%,
impurity B content 0.5%,
impurity C content less than 0.1%,
impurity D content less than 0.1%,
impurity F content less than 0.1%,
impurity G content less than 0.1%,
impurity H content less than 0.1% sum of other unidentified related oligosaccharides: approximately 0.4%.

A second purification by anion exchange chromatography is then carried out as described in example 2, step B. At the end of this step, a highly pure fondaparinux sodium composition which has the following characteristics is obtained:
Sodium fondaparinux content 99.7%,
impurity A content less than 0.1%,
impurity B content 0.3%,
impurity C content less than 0.1%,
impurity D content less than 0.1%,
impurity F content less than 0.1%,
impurity G content less than 0.1%,
impurity H content less than 0.1%
sum of other unidentified related oligosaccharides:
less than 0.2%.
   Cumulative Yield: 90.1%.

### EXAMPLE 4

Obtaining a Composition According to the Invention

Step A: Purification of the Crude Fondaparinux Sodium—Dynamic Treatment on Activated Charcoal

For the dynamic treatment, the preparation of the solution containing crude fondaparinux sodium is identical to that described in example 1, step A. The solution of fondaparinux sodium used in this example has a fondaparinux sodium concentration of 30 to 35 g per litre of water for injectable preparation. The pH is adjusted to 7.1 and the conductivity of the solution is adjusted to between 20 and 30 mS/cm.

The activated charcoal is, in this case, immobilized in a filter, using a resin-type adjuvant, between two cellulose plates (CUNO filter). The flow rate for percolation through the filter is from 100 to 500 1/h/M². The solution is recycled for two hours.

The washing is carried out with an NaCl solution having a conductivity of 20 to 35 mS/cm and a volume corresponding to 150% of the initial volume used. The washing is carried out continuously, without recirculation over the filter.

Next, the primary filtrate and also the washing solution are pooled and filtered through a membrane with a porosity of less than 1 μm (0.22 μm).

The batch of crude fondaparinux sodium used for this test contains 73.6% of fondaparinux sodium.

The amount of impurity A is equal to 2.7%,

the amount of impurity B is equal to 3.4%,
the amount of impurity C is equal to 0.9%,
the amount of impurity D is equal to 0.5%,
the amount of impurity F is equal to 0.4%,
the amount of impurity G is equal to 0.2%,
and the amount of impurity H is 0.3%.
   Amount of other unidentified related oligosaccharides: approximately 18%.

After the treatment on activated charcoal, the fondaparinux sodium composition is as follows:
Fondaparinux sodium content 89.6%,
impurity A content less than 0.2%,
impurity B content 2.5%,
impurity C content 0.7%,
impurity D content less than 0.2%,
impurity F content less than 0.1%,
impurity G content less than 0.1%,
impurity H content less than 0.1%,
yield: 97%.
   Step B: Purification by Anion Exchange Chromatography

The procedure as described in example 1, step B is then carried out. After analysis of the fractions, those having a purity of greater than 70% are pooled.

At the end of this step, a highly pure fondaparinux sodium composition which has the following characteristics is obtained:
Fondaparinux sodium content 97%,
impurity A content less than 0.1%,
impurity B content 0.9%,
impurity C content 0.2%,
impurity D content less than 0.1%,
impurity F content less than 0.1%,
impurity G content less than 0.1%,
impurity H content less than 0.1%,
sum of other unidentified related oligosaccharides: approximately 2%.
   Cumulative Yield: 94%.

A second purification by anion exchange chromatography is then carried out as described in example 2, step B. At the end of this step, a highly pure fondaparinux sodium composition which has the following characteristics is obtained:
Fondaparinux sodium content 99%,
impurity A content less than 0.1%,
impurity B content 0.2%,
impurity C content less than 0.1%,
impurity D content less than 0.1%,
impurity F content less than 0.1%,
impurity G content less than 0.1%,
impurity H content less than 0.1%,
sum of other unidentified related oligosaccharides: approximately 0.8%.
   Cumulative Yield: 87.7%.

The purified solutions containing fondaparinux sodium are concentrated so as to obtain solutions containing 20 to 70 g/l of fondaparinux sodium. The conductivity is adjusted with water or NaCl so as to be from 45 to 90 mS/cm. The solution thus obtained is filtered through a microfiltration membrane, and mixed with ethanol, at a ratio of 1:5 V/V.

A highly pure fondaparinux sodium composition having the following characteristics is thus obtained by precipitation:

Fondaparinux sodium content greater than 99.1%,

impurity A content less than 0.1%,
impurity B content less than 0.2%,
impurity C content less than 0.3%,
impurity D content less than 0.1%,
content of impurities F, G and H less than 0.1%,
cumulative yield: 78%.

1. A composition containing at least 97% but less than 100% of fondaparinux sodium, the remainder being related oligosaccharides.

2. A composition according to claim 1 containing at least 98% of fondaparinux sodium.

3. A purified fondaparinux sodium composition according to claim 2 containing not more than 0.8% of an oligosaccharide of formula III or of a mixture thereof with an oligosaccharide of formula IV 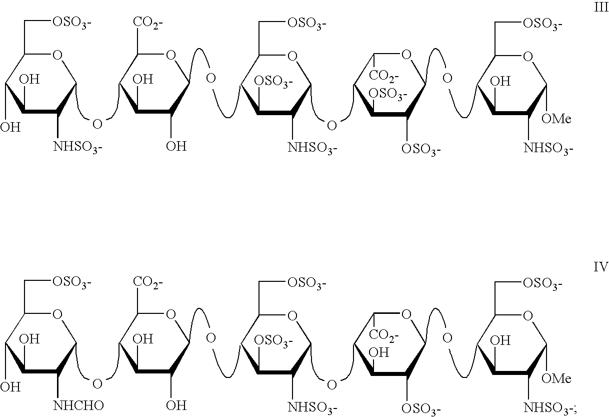
not more than 1.0% of an oligosaccharide of formula VI 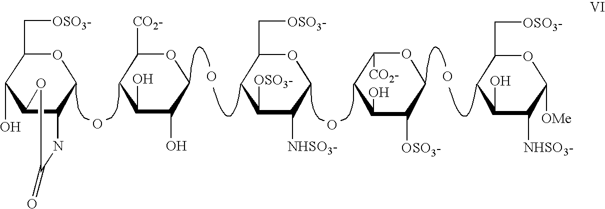
and not more than 1.0% of an oligosaccharide of formula V

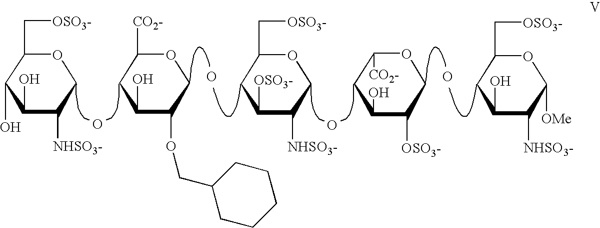

4. A purified fondaparinux sodium composition according to claim 2 containing no more than 0.8% of an oligosaccharide of formula III or of a mixture thereof with an oligosaccharide of formula IV 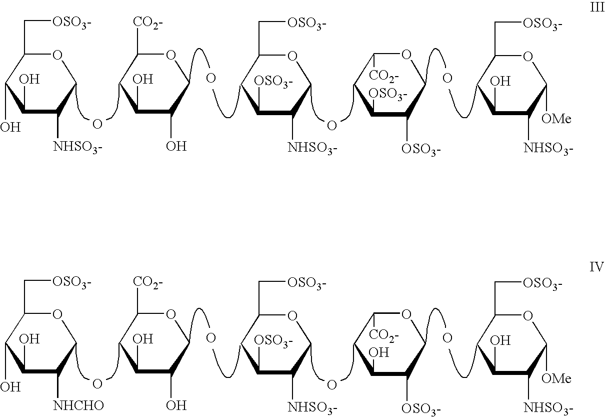
and not more than 0.6% of an oligosaccharide of formula V 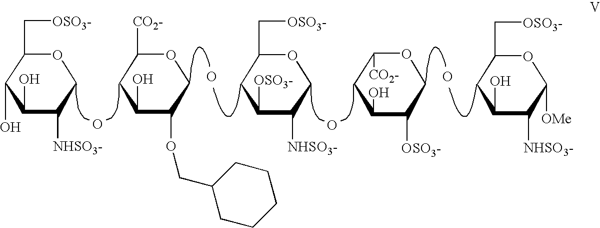
and wherein any oligosaccharide other than those of formulas III, IV and V is present in an amount not greater than 0.3%

5. A pharmaceutical composition comprising purified fondaparinux sodium according to claim 1 together with a pharmaceutically acceptable excipient.

6. A pharmaceutical composition comprising purified fondaparinux sodium according to claim 2 together with a pharmaceutically acceptable excipient.

7. A pharmaceutical composition comprising purified fondaparinux sodium according to claim 3 together with a pharmaceutically acceptable excipient.

8. A pharmaceutical composition comprising purified fondaparinux sodium according to claim 4 together with a pharmaceutically acceptable excipient.

9. A process for the preparation of a composition according to claim 1 which includes a step of treating impure fondaparinux sodium with activated charcoal.

10. A process according to claim 9 wherein an aqueous solution of crude fondaparinux sodium is passed through a bed of activated charcoal.

11. A process according to claim 10 wherein the activated charcoal is of plant origin and activated by steam.

12. A process according to claim 10 which additionally includes a step of purification by column chromatography.

13. A process according to claim 10 which additionally includes a step of precipitation with ethyl alcohol.

14. A process for the preparation of a composition according to claim 3 which includes a step of purification by treatment with activated charcoal.

15. A process according to claim 14 wherein an aqueous solution of crude fondaparinux sodium is passed through a bed of activated charcoal.

16. A process according to claim 15 which additionally includes a step of purification by column chromatography.

17. A process according to claim 15 which additionally includes a step of precipitation with ethyl alcohol.

18. A process for the preparation of a composition according to claim 4 which includes a step of purification by treatment with activated charcoal.

19. A process according to claim 18 wherein an aqueous solution of crude fondaparinux sodium is passed through a bed of activated charcoal.

20. A process according to claim 19 which additionally includes a step of purification by column chromatography.

21. A process according to claim 20 which additionally includes a step of purification with ethyl alcohol.

22. A process for the preparation of a composition containing at least 97% but less than 100% of fondaparinux sodium; not more than 0.8% of an oligosaccharide of formula III or of a mixture thereof with an oligosaccharide of formula IV 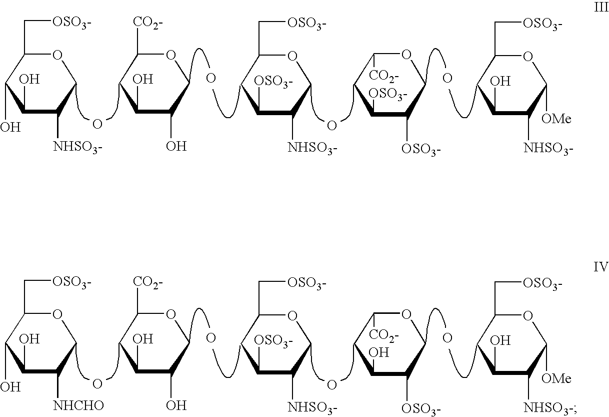
and not more than 1.0% of an oligosaccharide of formula VI 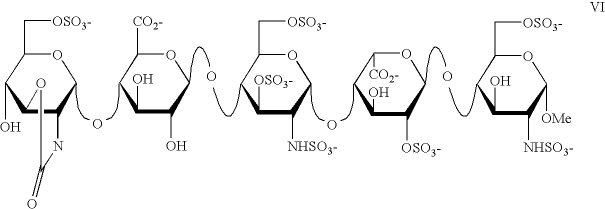
which includes a step of purification by treatment with activated charcoal.

23. A process according to claim 22 wherein an aqueous solution of crude fondaparinux sodium is passed through a bed of activated charcoal.

24. A process according to claim 23 which additionally includes a step of purification by column chromatography.

25. A process according to claim 23 which additionally includes a step of precipitation with ethyl alcohol.

26. A process according to claim 22 for the preparation of a composition containing at least 98% but less than 100% of fondaparinux sodium; not more than 0.5% of an oligosaccharide of formula III or a mixture thereof with an oligosaccharide for formula IV; not more than 0.3% of an oligosaccharide of formula VI; and wherein the amount of any oligosaccharide present in the composition other than those of formulas III, IV, and VI is not greater than 0.3%

27. A process according to claim 26 wherein an aqueous solution of crude fondaparinux sodium is passed through a bed of activated charcoal.

28. A process according to claim 27 which additionally includes a step of purification by column chromatography.

29. A process according to claim 27 which additionally includes a step of precipitation with ethyl alcohol.
